(19) United States
(12) Patent Application Publication
(10) Pub. No.: US 2005/0154028 A1
(43) Pub. Date: Jul. 14, 2005

(54) Cyclic urea derivatives with 5-ht2c receptor activity

(76) Inventors: Steven M Bromidge, Harlow, Essex (GB); Peter J. Lovell, Harlow, Essex (GB); Caroline Goodacre, Harlow, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/501,317

(22) PCT Filed: Jan. 7, 2003
(86) PCT No.: PCT/GB03/00020

(30) Foreign Application Priority Data
Jan. 8, 2002 (GB) 0200283.0

(51) Int. Cl. A61K 31/4439; A61K 31/4196; A61K 31/4166; C07D 249/12; C07D 233/66; C07D 43/04

(52) U.S. Cl. 514/341; 514/384; 514/389; 546/272.4; 546/272.7; 548/263.2; 548/316.4

(57) ABSTRACT

Compounds of formula (I) or a pharmaceutically acceptable salt thereof, having 5HT_{2C} receptor activity, are disclosed: wherein a is 0,1,2,3,4 or 5; b is 1,2 or 3; Y is nitrogen or carbon; A is oxygen, nitrogen, —CONH—, —NHCO— or together with R² form a benzoxazolone group; R¹ is halogen, C₁₋₆alkyl, C₁₋₆alkoxy, C₁₋₆alkylthio, hydroxy, amino, mono- or di-C₁₋₆alkylamino, nitro, CN, CF₃, OCF₃, aryl, arylC₁₋₆alkyl, arylC₁₋₆alkyloxy or arylC₁₋₆alkylthio; R² is hydrogen, halogen, C₁₋₆alkyl, C₁₋₆alkoxy, haloC₁₋₆alkyl or haloC₁₋₆alkoxy; R³ is: (i) —NR⁴R⁵ where R⁴ and R⁵ are independently hydrogen, C₁₋₆alkyl or arylC₁₋₆alkyl; or (ii) an optionally substituted N-linked heterocycle; or (iii) an optionally substituted C-linked heterocycle; ======= is a single bond or a double bond; and X is CH₂ or C═O (when ======= is a single bond) or X is CH (when ======= is a double bond). The preparation of these compounds and their use in therapy, especially for a CNS disorder such as depression or anxiety, are also disclosed.

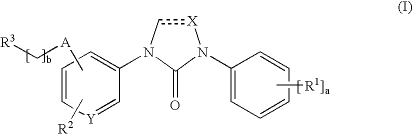

This invention relates to novel compounds having pharmacological activity, processes for their preparation, to compositions containing them and to their use in the treatment and/or prevention of CNS and other disorders.

WO 96/23783, WO 97/46699 and WO 97/48700 all disclose a series of indoline derivatives which are 5-HT_{2C} receptor antagonists and which are claimed to be useful in the treatment of various CNS disorders.

A novel class of compounds possessing 5-HT_{2C} receptor activity has been found. The present invention therefore provides, in a first aspect, a compound of formula (I) or a pharmaceutically acceptable salt thereof: 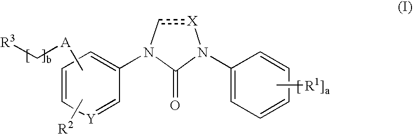
wherein:
a is 0,1,2,3,4 or 5;
b is 1, 2 or 3;
Y is nitrogen or carbon;
A is oxygen, nitrogen, —CONH—, —NHCO— or together with R² form a benzoxazolone group;
R¹ is halogen, C₁₋₆alkyl, C₁₋₆alkoxy, C₁₋₆alkylthio, hydroxy, amino, mono- or di-C₁₋₆alkylamino, nitro, CN, CF₃, OCF₃, aryl, arylC₁₋₆alkyl, arylC₁₋₆alkyloxy or arylC₁₋₆alkylthio;
R² is hydrogen, halogen, C₁₋₆alkyl, C₁₋₆alkoxy, haloC₁₋₆alkyl or haloC₁₋₆alkoxy;
R³ is: (i)-NR⁴R⁵ where R⁴ and R⁵ are independently hydrogen, C₁₋₆alkyl or arylC₁₋₆alkyl; or
   (ii) an optionally substituted N-linked heterocycle; or
   (iii) an optionally substituted C-linked heterocycle; 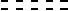
      is a single bond or a double bond;
X is CH₂ or C═O (when 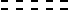
   is a single bond) or X is CH (when 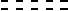
   is a double bond).

The following terms, whether used alone or as part of another group are to be given the following meanings, unless otherwise stated.

The term “halogen” and its abbreviated form “halo” are used herein to describe fluorine, chlorine, bromine or iodine.

The term “alkyl” is used herein to describe a straight chain or branched fully saturated hydrocarbon group. “C₁₋₆alkyl” refers to alkyl groups having from one to six carbon atoms, including all isomeric forms, such as methyl, ethyl, propyl, isopropyl, butyl, isobutyl, sec-butyl, tert-butyl, pentyl, neopentyl, sec-pentyl, n-pentyl, isopentyl, tert-pentyl and hexyl.

The term “C₁₋₆alkoxy” refers to a straight chain or branched chain alkoxy (or “alkyloxy”) group having from one to six carbon atoms, including all isomeric forms, such as methoxy, ethoxy, propoxy, isopropoxy, butoxy, isobutoxy, sec-butoxy, tert-butoxy, pentoxy, neopentoxy, sec-pentoxy, n-pentoxy, isopentoxy, tert-pentoxy and hexoxy.

The term “C₁₋₆alkylthio” refers to a straight chain or branched chain alkylthio group having from one to six carbon atoms, such as methylthio, ethylthio, propylthio, isopropylthio, butylthio, isobutylthio, sec-butylthio, tert-butylthio, pentylthio, neopentylthio, sec-pentylthio, n-pentylthio, isopentylthio, tert-pentylthio and hexylthio.

The term “mono- or di-C₁₋₆alkylamino” refers to an amino group which is substituted by one C₁₋₆alkyl group or an amino group which is substituted by two C₁₋₆alkyl groups, the two amino groups being the same or different. Examples of monoC₁₋₆alkylamino include methylamine, ethylamine, propylamine, isopropylamine, butylamine, isobutylamine, sec-butylamine, tert-butylamine, pentylamine, neopentylamine, sec-pentylamine, n-pentylamine, isopentylamine, tert-pentylamine and hexylamine. Examples of di-C1-6alkylamino include dimethylamine, diethylamine, dipropylamine, diisopropylamine, dibutylamine, diisobutylamine, disec-butylamine, ditert-butylamine, dipentylamine, dineopentylamine, dihexylamine, butylmethylamino, isopropylmethylamino, ethylisopropylamino, ethylmethylamino, etc.

The term “aryl” is used herein to describe groups such as phenyl or naphthyl, which may be optionally substituted by one or more of C₁₋₆alkyl (to form “arylC₁₋₆alkyl”), halogen, CF₃ or C₁₋₆alkoxy (to form “arylC₁₋₆alkoxy”).

The terms “halo C₁₋₆alkoxy” or “haloC₁₋₆alkyl” are used to describe a C₁₋₆alkoxy or a C₁₋₆alkyl group, respectively, substituted with one or more halogens. Examples include —CHCl₂, —CF₃, —OCF₃, etc.

The term “optionally substituted N-linked heterocycle” is used herein to describe a stable non-aromatic 5-7 membered ring containing at least 1 nitrogen atom and optionally a further 1 or 2 heteroatoms selected from nitrogen, sulphur or oxygen, wherein the heterocycle is linked to the remainder of the molecule via a nitrogen atom.

The term “optionally substituted C-linked heterocycle” is used herein to describe a stable non-aromatic 5-7 membered ring containing at least 1 nitrogen atom and optionally a further 1 or 2 heteroatoms selected from nitrogen, sulphur and oxygen, wherein the heterocycle is linked to the remainder of the molecule via a carbon atom.

Suitable examples of N-linked or C-linked heterocycles include pyrrolidinyl, piperazinyl, morpholinyl, imidazolidinyl, thiomorpholinyl, piperidinyl and azepanyl.

More than one optional substituent may be present in the N-linked or C-linked heterocycle, which may be the same or different, and may be attached to any carbon atom of the heterocycle or an available nitrogen atom.

Suitable optional substituents for the N-linked or C-linked heterocycle include C₁₋₆alkyl, amino, mono- or di- C₁₋₆alkylamino, aryl, arylC₁₋₆alkyl, arylamino, hydroxy, C₁₋₆alkylamido, hydroxyC₁₋₆alkyl, C₁₋₆alkoxycarbonyl, halogen, haloC₁₋₆alkyl, a heteroaromatic group (such as indole or benzimidazole), an aromatic or non-aromatic N-linked or C-linked heterocycle or an aromatic or non-aromatic heterocycleC₁₋₆alkyl optionally substituted by C₁₋₆alkyl. Examples of aromatic or non-aromatic heterocycleC₁₋₆alkyl include heterocycle-methyl (such as pyridinyl-methyl and benzimidazolyl-methyl) and heterocycle-ethyl (such as morpholinyl-ethyl and indolyl-ethyl).

Substituents in the N-linked or C-linked heterocycle may form a bridge structure, to form a group such as for example 2-oxa-5-azabicyclo[2.2.1]heptyl. Such a bicyclic group may be further substituted by the substituents listed above. More than one substituent may be present on the same carbon atom to form spiro structures such as 1,4 and 1,5 dioxa spiro compounds.

When a is 2 or more, the 2 or more R¹ groups may be the same or different.

When A is nitrogen, A and R² may be joined to form a benzoxazolone group as follows:

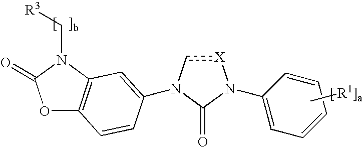

Preferred compounds of the present invention include those in which, independently or in any compatible combination:
a is 1 or 2;
R¹ is halogen, particularly fluoro or chloro;
R¹ is at the 3 or 4 position, ie at the meta and/or para positions of the benzene to which R¹ is attached;
X is CH₂ and 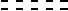
   represents a single bond, or X is CH and 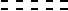
   represents a double bond;
Y is carbon;
R² is at the following position: 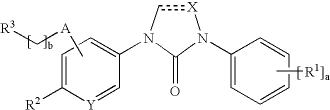
R² is C₁₋₆alkoxy, particularly methoxy;
A is at the following position: 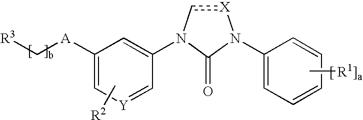
A is oxygen;
b is2;
R³ is an N-linked heterocycle, particularly piperidinyl, or is a diC₁₋₆alkylamine, particularly dimethylamine.

Exemplary compounds of this invention include:
1-(3-Fluoro-phenyl)-3-[4-methoxy-3-(2-piperidin-1-yl-ethoxy)-phenyl]-imidazolidin-2-one
1-(3-Fluoro-phenyl)-3-[4-methoxy-3-(2-morpholin-4-yl-ethoxy)-phenyl]-imidazolidin-2-one
1-(4-Methyl-3-trifluoromethyl-phenyl)-3-[4-methoxy-3-(2-piperidin-1-yl-ethoxy)-phenyl]-imidazolidin-2-one
1-(4-Methyl-3-trifluoromethyl-phenyl)-3-[4-methoxy-3-(2-morpholin-4-yl-ethoxy)-phenyl]-imidazolidin-2-one
1-(2-Chloro-phenyl)-3-[4-methoxy-3-(2-piperidin-1-yl-ethoxy)-phenyl]-imidazolidin-2-one
1-[4-Methoxy-3-(2-piperidin-1-yl-ethoxy)-phenyl]-3-phenyl-imidazolidin-2-one
1-[3-(2-Dimethylamino-ethoxy)₄-methoxy-phenyl]-3-(3-fluorophenyl)-imidazolidin-2-one
1-(3-Fluoro-phenyl)-3-{4-methoxy-3[(1S,4S)-2-(2-oxa-5-aza-bicyclo[2.2.1]hept-5-yl)-ethoxy]-phenyl}-imidazolidin-2-one
1-(3,4-Dichloro-phenyl)-3-[4-methoxy-3-(2-piperidin-1-yl-ethoxy)-phenyl]-imidazolidin-2-one
1-(3,4-Dichloro-phenyl)-3-[4-methoxy-3-(2-morpholin-4-yl-ethoxy)-phenyl)-imidazolidin-2-one
1-(3,4-Dichloro-phenyl)-3-[3-(2-dimethylamino-ethoxy)-4-methoxy-phenyl]-imidazolidin-2-one
1-(3,4-Dichloro-phenyl)-3-[6-(2-piperidin-1-yl-ethoxy)-pyridin-3-yl]-imidazolidin-2-one
1-[4-Bromo-3-(2-piperidin-1-yl-ethoxy)-phenyl]-3-(3,4-dichloro-phenyl)-imidazolidin-2-one
1-(2,3-Dichloro-phenyl)-3-[4-methoxy-3-(2-piperidin-1-yl-ethoxy)-phenyl]-imidazolidin-2-one
1-(3-Fluoro-phenyl)-3-[4-methoxy-3-(2-piperidin-1-yl-ethoxy)-phenyl]-1,3-dihydro-imidazol-2-one
1-(3-Fluoro-phenyl)-3-[4-methoxy-3-(2-piperidin-1-yl-ethoxy)-phenyl]-1,3-dihydro-imidazol-2-one
1-(3-Fluoro-phenyl)-3-[4-methoxy-3-(2-piperidin-1-yl-ethoxy)-phenyl]-imidazol-2-one
3-(3-Fluoro-phenyl)-1-[4-methoxy-3-(2-piperidin-1-yl-ethoxy)-phenyl]-imidazol-2,4-dione
5-[3-(3,4-Dichloro-phenyl)-2-oxo-imidazolidin-1-yl]-3-(2-piperidin-1-yl-ethyl)-3H-benzoxazol-2-one
   and pharmaceutically acceptable salts thereof.

The compounds of formula (I) can form acid addition salts. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid.

The compounds of this invention may be in crystalline or non-crystalline form, and, if crystalline, may optionally be hydrated or solvated. This invention includes within its scope stoichiometric hydrates as well as compounds containing variable amounts of water.

Certain compounds of formula (I) are capable of existing in stereoisomeric forms (e.g. geometric or (“cis-trans”) isomers, diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The present invention also provides a process for the preparation of a compound of formula (I) or a pharmaceutically acceptable salt thereof, which process comprises:
a) the cyclisation of a compound of formula (II) 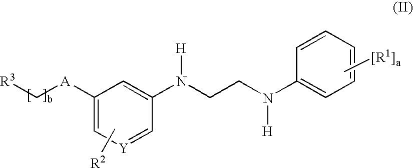
in which R¹, R², R³, A, Y, a and b are defined in formula (I); or
b) the coupling of a compound of formula (III): 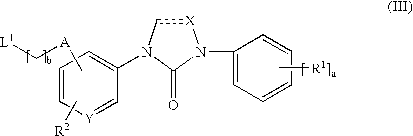
in which R¹, R², A, X, Y, a and b are defined in formula (I) and L¹ is a leaving group, with a compound of formula (IV):
   H—NR⁴R⁵ (IV)
in which R⁴ and R⁵ are defined in formula (I); or
c) the coupling of a compound of formula (V)= 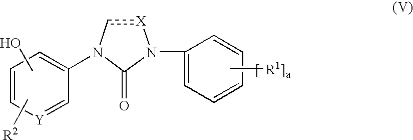
in which R¹, R², X, Y and a are defined in formula (I), with a compound of formula (VI): 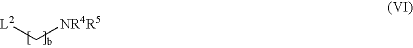
in which R⁴, R⁵ and b are defined in formula (I) and L² is a leaving group or hydroxy; or
d) the cyclisation of a compound of formula (VII) 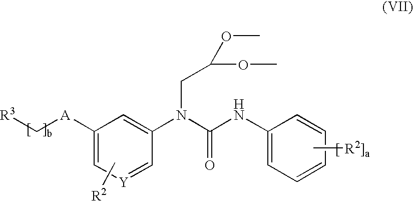
in which R¹, R², R³, Y, A, a and b are defined in formula (I);
optionally followed by:
   removing any protecting groups; and/or
   converting a compound of formula (I) into another compound of formula (I); and/or
   forming a pharmaceutically acceptable salt.

For process a), suitable cyclising agents include phosgene, triphosgene and CDI (preferably phosgene). The cyclisation reaction of compounds of formula (II) is preferably carried out in an inert solvent such as tetrahydrofuran in the presence of a base such as triethylamine.

For process b), suitably L¹ is mesylate, tosylate, bromo or chloro (preferably mesylate). The reaction of compounds of formulae (III) and (IV) are typically carried out in the presence of a base such as potassium carbonate or sodium carbonate (preferably potassium carbonate) in a suitable solvent such as N,N-dimethylformamide.

For process c), suitably L² is halogen (preferably chloro) or hydroxy. When L² is halogen, the reaction of compounds of formulae (V) and (VI) are typically carried out in the presence of a base such as potassium carbonate or sodium carbonate (preferably potassium carbonate) in a suitable solvent such as ethylene glycol dimethyl ether. When L² is hydroxy, suitable Mitsunobu reagents include triphenylphosphine and DEAD in a suitable solvent such as tetrahydrofuran.

For process d), suitable cyclising conditions include catalytic acid (preferably HCl) in an inert solvent such as toluene.

Compounds of formula (I) can be converted into further compounds of formula (I) using standard techniques. For example, and by way of illustration rather than limitation, compounds of formula (1) in which 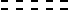
is a double bond can be converted to compounds of formula (I) in which 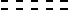
is a single bond by palladium catalysed hydrogenation in a suitable solvent such as ethanol.

Compounds of formulae (IV) and (VI) are commercially available. Compounds of formulae (II), (III), (V) and (VII) may be prepared according to methods described herein, or may be prepared according to known methods or by analogous methods thereto.

For example, a compound of formula (II) may be prepared by reacting a compound of formula (VIII): 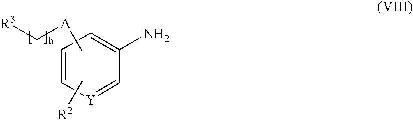
with a compound of formula (IX): 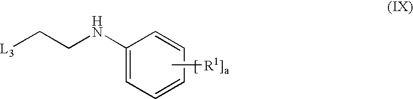
wherein L₃ is a leaving group.

A compound of formula (III) may be prepared by reacting a compound of formula (V) with a compound of formula (X): 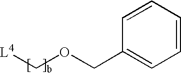
(X)wherein L⁴ is a leaving group, followed by subsequent debenzylation and activation of the alcohol to a leaving group (L¹).

A compound of formula (V) may be prepared by the phosgene cyclisation of a compound of formula (XI) 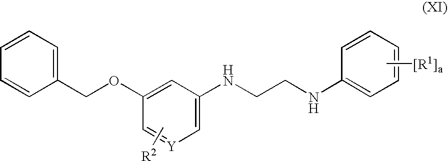
followed by subsequent debenzylation.

A compound of formula (VII) may be prepared for example by reacting a compound of formula (XII: 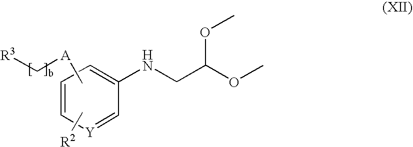
with phenyl isocyanate having the desired [R¹] a substituents. Compounds of formula (XII) may be prepared for example by reacting a compound of formula (VIII) with glyoxal 1,1-dimethyl acetal under reducing conditions, such as catalytic hydrogenation over a palladium catalyst.

Those skilled in the art will appreciate that it may be necessary to protect certain groups to carry out the above processes. Suitable protecting groups and methods for their attachment and removal are conventional in the art of organic chemistry, such as those described in Greene T. W. ‘Protective groups in organic synthesis’ New York, Wiley (1981).

Pharmaceutically acceptable salts may be prepared conventionally by reaction with the appropriate acid or acid derivative.

In another aspect, the present invention provides a pharmaceutical composition comprising a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salt thereof and a sterile vehicle. Formulations for injection may be presented in unit dosage form e.g. in ampoules or in multi-dose, utilising a compound of the invention or pharmaceutically acceptable salt thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa butter or other glycerides.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unitary dose device or alternatively as a powder mix with a suitable carrier for administration using a suitable delivery device. Thus compounds of formula (I) may be formulated for oral, buccal, parenteral, topical (including ophthalmic and nasal), depot or rectal administration or in a form suitable for administration by inhalation or insufflation (either through the mouth or nose).

The compounds of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

Compounds of formula (I) and their pharmaceutically acceptable salts are of use in the treatment of certain CNS disorders such as depression, which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, vascular dementia with depressed mood, schizoaffective disorder or the depressed type, and depressive disorders resulting from general medical conditions including, but not limited to, myocardial infarction, diabetes, miscarriage or abortion, etc. Other CNS disorders which may be treated or prevented include anxiety disorders, including generalised anxiety, schizophrenia, panic disorder, agoraphobia, social phobia, obsessive compulsive disorder and post-traumatic stress disorder, pain (particularly neuropathic pain), memory disorders, including dementia, amnesic disorders and age-associated memory impairment, disorders of eating behaviours, including anorexia nervosa and bulimia nervosa, sexual dysfunction, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), sedative ipnotic, amphetamine or amphetamine-related drugs (e.g. dextroamphetamine, methylamphetamine) or a combination thereof, motor disorders such as Parkinson's disease, dementia in Parkinson's disease, neuroleptic-induced Parkinsonism and tardive dyskinesias, as well as other psychiatric disorders.

Compounds of formula (I) may also have utility in the treatment of certain gastrointestinal disorders such as irritable bowel syndrome, Crohn's disease, ulcerative colitis, non-steroidal anti-inflammatory drug induced damage.

As used herein, the term “treatment” refers to amelioration and/or cure of established symptoms as well as prophylaxis.

Thus the invention also provides a compound of formula (I) or a pharmaceutically acceptable salt thereof, for use as a therapeutic substance, in particular in the treatment of the above disorders. In particular the invention provides a compound of formula (I) or a pharmaceutically acceptable salt thereof for use as a therapeutic substance in the treatment of depression.

Compounds of the invention may be administered in combination with other active substances such as 5HT3 antagonists, NK-1 antagonists, serotonin agonists, selective serotonin reuptake inhibitors (SSRI), noradrenaline re-uptake inhibitors (SNRI), tricyclic antidepressants and/or dopaminergic antidepressants.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

It will be appreciated that the compounds of the combination or composition may be administered simultaneously (either in the same or different pharmaceutical formulations), separately or sequentially.

The invention further provides a method of treatment of the above disorders in mammals including humans, which comprises administering to the sufferer a therapeutically safe and effective amount of a compound of formula (I) or a pharmaceutically acceptable salt thereof.

In another aspect, the invention provides for the use of a compound of formula (I) or a pharmaceutically acceptable salt thereof in the manufacture of a medicament for use in the treatment of the above disorders.

The composition of the present invention may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 200 mg, and such unit doses may be administered more than once a day, for example two or three times a day. Such therapy may extend for a number of weeks or months. When administered in accordance with the invention, no unacceptable toxicological effects are expected with the compounds of the invention.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the present invention.

### DESCRIPTION 1

### 2-(3-Fluoro-phenylamino)-ethanol (D1)

To a solution of 3-fluoroaniline (5 g, 45 mmol) in dichloromethane (100 mL) and pyridine (4.6 mL, 57 mmol) at room temperature was slowly added 2-chloroethylchloroformate (4.6 mL, 45 mmol). The mixture was stirred at room temperature for 2.5 hours and washed with water (×4), dried over anhydrous magnesium sulphate and concentrated in vacuo. The residue was dissolved in ethanol (100 mL), treated with potassium hydroxide (10.1 g, 180 mmol) and heated at 90° C. for 4 hours. The mixture was concentrated in vacuo and the residue partitioned between dichloromethane (250 mL) and water (150 mL), The aqueous layer was re-extracted and the combined organics washed with water (×5) and brine. The organics were dried over anhydrous magnesium sulphate and concentrated in vacuo. The residue was purified by silica gel chromatography to give the title compound as an orange oil (5.6 g, 81%). MH⁺ 156.

### DESCRIPTION 2

### 2-(4-Methyl-3-trifluoromethyl-phenylamino)-ethanol (D2)

The title compound was prepared from 5-amino-2-methylbenzotrifluoride using the method described in Description 1. MH⁺ 220.

### DESCRIPTION 3

### 2-(2-Chloro-phenylamino)-ethanol (D3)

The title compound was prepared from 2-chloroaniline using the method described in Description 1. (M−H₂O+H)⁺ 154/156.

### DESCRIPTION 4

### 2-(3,4-Dichloro-phenylamino)-ethanol (D4)

The title compound was prepared from 3,4-dichloroaniline using the method described in Description 1. MH⁺ 206/208/210.

### DESCRIPTION 5

### 2-(2,3-Dichloro-phenylamino)-ethanol (D5)

The title compound was prepared from 2,3-dichloroaniline using the method described in Description 1. MH⁺ 206/208/210.

### DESCRIPTION 6

### 2-Benzyloxy-1-methoxy-4-nitro-benzene (D6)

A mixture of benzyl bromide (3.1 mL, 26.6 mmol), 2-methoxy-5-nitrophenol (3 g, 17.7 mmol) and sodium iodide (0.07 g, 0.5 mmol) in ethylene glycol dimethyl ether (100 mL), water (10 mL) and saturated potassium carbonate solution (35 mL) was heated at 65° C. for 23 hours. The mixture was concentrated in vacuo and the residue partitioned between ethyl acetate (200 mL) and saturated sodium bicarbonate solution (75 mL). The organic phase was washed with brine (125 mL, 100 mL), dried over anhydrous magnesium sulphate and concentrated in vacuo. The residue was purified by silica gel chromatography to give the title compound as a yellow solid (4.5 g, 98%).

### DESCRIPTION 7

### 3-Benzyloxy-4-methoxy-phenylamine (D7)

Iron powder (0.85 g, 15.2 mmol) was added to a suspension of D6 (1.7 g, 6.6 mmol) in methanol (75 mL) and saturated aqueous ammonium chloride (35 mL) and heated at reflux for 2 hours. The mixture was allowed to cool to room temperature and filtered through a plug of celite and washed with methanol. The methanol was removed in vacuo and the residue partitioned between ethyl acetate (125 mL) and water (enough to dissolve the ammonium chloride). The aqueous layer was re-extracted with ethyl acetate (50 mL) and the combined organics washed with brine (125 mL), dried over anhydrous magnesium sulphate and concentrated in vacuo. The residue was purified by silica gel chromatography to give the title compound as a brown solid (1.24 g, 83%). MH⁺ 230.

### DESCRIPTION 8

### 1-[2-(2-Methoxy-5-nitro-phenoxy)-ethyl]-piperidine (D8)

A mixture of 2-methoxy-5-nitrophenol (15 g, 88.8 mmol) and 1-(2-chloroethyl)piperidine monohydrochloride (21.2 g, 115 mmol) in ethylene glycol dimethyl ether (600 mL), water (226 mL) and saturated potassium carbonate solution (226 mL) was stirred at room temperature for 24 hours. The organic phase was separated, washed with water (400 mL), dried over anhydrous magnesium sulphate and concentrated in vacuo to give the title compound as brown crystals (24.6 g, 98%). MH⁺ 281.

### DESCRIPTION 9

### 4-Methoxy-3-(2-piperidin-1-yl-ethoxy)-phenylamine (D9)

D8 (24.6 g, 87.9 mmol) was dissolved in ethanol (500 mL). 10% Palladium on charcoal (2 g) was added and the reaction mixture hydrogenated at atmospheric pressure for 16 hours. The mixture was filtered through a small plug of celite and the solvent removed in vacuo to give the title compound as brown crystals (21.8 g, 100%). MH⁺ 251.

### DESCRIPTION 10

### 4-[2-(2-Methoxy-5-nitro-phenoxy)-ethyl]-morpholine (D10)

The title compound was prepared from 2-methoxy-5-nitrophenol and 4-(2-chloroethyl)morpholine monohydrochloride stirring for 48 hours using the method described in Description 8. MH⁺ 283.

### DESCRIPTION 11

### 4-Methoxy-3-(2-morpholin-4yl-ethoxy)-phenylamine (D11)

The title compound was prepared from D10 using the method described in Description 9. MH⁺ 253.

### DESCRIPTION 12

### [2-(2-Methoxy-5-nitro-phenoxy)-ethyl]-dimethyl-amine (D12)

Diethyl azodicarboxylate (4.6 mL, 29 mmol) was added dropwise to a stirred solution of 2-methoxy-5-nitrophenol (4.97 g, 29 mmol), triphenylphosphine (7.7 g, 29 mmol) and N,N-dimethylethanolamine (2.95 mL, 29 mmol) at room temperature. After stirring for 48 hours the solvent was removed in vacuo and the residue partitioned between ethyl acetate (200 mL) and saturated sodium bicarbonate solution (125 mL). The organic phase was washed with brine (2×100 mL), dried over anhydrous magnesium sulphate and concentrated in vacuo. The residue was purified by silica gel chromatography to give the title compound as a yellow solid (1.99 g, 28%). MH⁺ 241.

### Description 13

### 3-(2-Dimethylamino-ethoxy)-4-methoxy-phenylamine (D13)

The title compound was prepared from D12 using the method described in Description 9.

### DESCRIPTION 14

### 5-Nitro-2-(2-piperidin-1-yl-ethoxy)-pyridine (D14)

N-(2-Hydroxyethyl) piperidine (2.44 g, 18.9 mmol) was dissolved in N,N-dimethylformamide (40 mL), cooled to 0° C. in an ice/water bath and sodium hydride (60% dispersion in oil, 0.682 g, 17 mmol) added portionwise over ten minutes. After stirring for 35 minutes at 0° C. the reaction mixture was allowed to warm to room temperature and was stirred for 2 hours. The mixture was cooled to 0° C. and 2-chloro-5-nitropyridine (3.0 g, 18.9 mmol) added portionwise. The mixture was allowed warm to room temperature and stirred for 16 hours. The solvent was removed in vacuo and the residue partitioned between ethyl acetate (150 mL) and saturated sodium bicarbonate solution (250 mL). The organic phase was washed with brine (150 mL), dried over anhydrous magnesium sulphate and concentrated in vacuo. The residue was purified by silica gel chromatography to give the title compound as a brown solid (0.897 g, 19%). MH⁺ 252.

### DESCRIPTION 15

### 6-(2-Piperidin-1-yl-ethoxy)-pyridin-3-ylamine (D15)

D14 (0.435 g, 1.7 mmol) was dissolved in ethanol (50 mL). 10% Palladium on charcoal (0.15 g) was added and the reaction mixture hydrogenated at atmospheric pressure for 2 hours and 20 minutes. The mixture was filtered through a small plug of celite and the solvent removed in vacuo to give the title compound as a grey solid (0.373 g, 97%).

### DESCRIPTION 16

### N-(4-Bromo-3-hydroxy-phenyl)-acetamide (D16)

3-Acetamidophenol (45.5 g, 0.3 mol) in acetic acid (400 mL) was treated with bromine (15.5 ml, 0.3 mol) in acetic acid (100 mL) at room temperature with stirring. The mixture was stirred at room temperature for 60 hours and filtered to give the title product as a white solid (67.55 g, 97%). MH⁻ 228/230.

### DESCRIPTION 17

### N-[4-Bromo-3-(2-piperidin-1-yl-ethoxy)-phenyl]-acetamide (D17)

A mixture of D16 (4.6 g, 20 mmol) and 1-(2-chloroethyl)piperidine monohydrochloride (4.05 g, 22 mmol) in ethylene glycol dimethyl ether (200 mL), water (50 mL) and saturated potassium carbonate solution (50 mL) was stirred at 70° C. for three hours and at room temperature for 16 hours. The organic phase was separated, the aqueous layer extracted with dichloromethane, and the combined organics dried over anhydrous magnesium sulphate. The solvent was removed in vacuo to give the title compound as a white solid (4.2 g, 62%). MH⁺ 341/343.

### DESCRIPTION 18

### 4-Bromo-3-(2-piperidin-1-yl)-ethoxy)-phenylamine (D18)

A solution of D17 (2 g, 5.9 mmol) in ethanol (20 mL) and 40% sodium hydroxide solution (20 mL) was heated at 80° C. for two hours. The reaction mixture was cooled and the organic layer separated and concentrated in vacuo. The residue was partitioned between dichloromethane and water and the organic layer dried over anhydrous magnesium sulphate. The solvent was removed in vacuo to give the title compound as a yellow solid (1.7 g, 97%). MH⁺ 298/300

### DESCRIPTION 19

### (2,2-Dimethoxy-ethyl)-[4-methoxy-3-(2-piperidin-1-yl-ethoxy)-phenyl]-amine (D19)

To a solution of D9 (2.49 g, 10 mmol) and glyoxal 1,1-dimethyl acetal solution (˜45% in tert-butyl methyl ether, 2.8 ml, 12 mmol) in ethanol (50 mL) was added 10% palladium/carbon (0.45 g) and the mixture hydrogenated at atmospheric pressure for 31 hours. The mixture was filtered through a small plug of celite and the solvent removed in vacuo. The residue was purified by silica gel chromatography to give the title compound as a brown oil (1.81 g, 54%). MH⁺ 339.

### DESCRIPTION 20

### 1-(2,2-Dimethoxy-ethyl)-3-(3-fluoro-phenyl)-1-[4-methoxy-3-(2-piperidin-1-yl-ethoxy)-phenyl]-urea (D20)

3-Fluorophenyl isocyanate (0.186 g, 1.4 mmol) in dichloromethane (1 mL) was added to a solution of D19 (0.455 g, 1.3 mmol) in dichloromethane (15 mL) at room temperature. After stirring for 16 hours at room temperature the reaction mixture was diluted with dichloromethane (45 mL), washed with saturated sodium bicarbonate solution (20 mL) and brine (50 mL). The organic phase was dried over anhydrous magnesium sulphate and the solvent removed in vacuo. The residue was purified by silica gel chromatography to give the title compound as a white solid (0.316 g, 49%). MH⁺ 476.

### DESCRIPTION 21

### 3-(3,5-Difluoro-phenyl)-1-(2,2-dimethoxy-ethyl)-1-[4-methoxy-3-(2-piperidin-1-yl-ethoxy)-phenyl]-urea

The title compound was prepared from 3,5-difluorophenyl isocyanate and D19 using the method described in Description 20. MH⁺ 494.

### DESCRIPTION 22

### (3-Benzyloxy-4-methoxy-phenylamino)-acetic Acid Ethyl Ester (D22)

D7 (1.5 g, 6.6 mmol) was dissolved in N,N-dimethylformamide (20 mL) and treated with potassium carbonate (1.81 g, 13.1 mmol), ethyl bromoacetate (0.73 mL, 6.6 mmol) and sodium iodide (0.983 g, 6.6 mmol). The reaction mixture was heated at 80° C. for 17 hours and allowed to stand at room temperature for 48 hours. The solvent was removed in vacuo and the residue partitioned between between ethyl acetate (125 mL) and half saturated sodium bicarbonate solution (85 mL). The organic phase was washed with brine (150 mL), dried over anhydrous magnesium sulphate and concentrated in vacuo. The residue was purified by silica gel chromatography to give the title compound (1.3 g, 63%). MH⁺ 316.

### DESCRIPTION 23

### 1-(3-Benzyloxy-4-methoxy-phenyl)-3-(3-fluoro-phenyl)-imidazolidine-2,4-dione (D23)

D22 (1.3 g, 4.1 mmol) was dissolved in toluene (50 mL) and 3-fluorophenyl isocyanate (0.566 g, 4.1 mmol) added. The reaction mixture was heated at 85° C. for ten hours. The solvent was removed in vacuo and the residue purified by silica gel chromatography to give the title compound as a pale yellow solid (1.03 g, 61%). MH⁺ 407.

### DESCRIPTION 24

### N-(3-Benzyloxy-4-methoxy-phenyl)-N′-(3-fluoro-phenyl)-ethane-1,2-diamine (D24)

Methanesulfonyl chloride (0.68 mL, 8.8 mmol) was added dropwise to a solution of D1 (1.36 g, 8.8 mmol) in dichloromethane (17 mL) and triethylamine (1.83 mL, 13.2 mmol) at room temperature and stirred for 30 minutes. The mixture was diluted with dichloromethane (120 mL) and washed with water (75 mL) and brine (115 mL). The organic phase was dried over anhydrous magnesium sulphate and concentrated in vacuo. The residue and sodium iodide (1.45 g, 9.7 mmol) were added at 50° C. to a mixture of D7 (2.3 g, 10 mmol) and potassium carbonate (1.22 g, 8.8 mmol) in N,N-dimethylformamide (17 mL). The reaction mixture was heated at 80° C. for 16 hours and allowed to cool to room temperature. The solvent was removed in vacuo and the residue partitioned between ethyl acetate (160 mL) and water (100 mL). The organic phase was washed with brine (175 mL), dried over anhydrous magnesium sulphate and concentrated in vacuo. The residue was purified by silica gel chromatography to give the title compound as a brown oil (1.5 g, 47%). MH⁺ 367.

### DESCRIPTION 25

### N-(3-Benzyloxy-4-methoxy-phenyl)-N′-(4-methyl-3-trifluoromethyl-phenyl)-ethane-1,2-diamine (D25)

The title compound was prepared from D2 and D7 using the method described in Description 24. MH⁺ 431.

### DESCRIPTION 26

### N-(3-Benzyloxy-4-methoxy-phenyl)-N′-(2-chloro-phenyl)-ethane-1,2-diamine (D26)

The title compound was prepared from D3 and D7 using the method described in Description 24. MH⁺ 382/384

### DESCRIPTION 27

### N-(3,4-Dichloro-phenyl)-N′-[4-methoxy-3-(2-piperidin-1-yl-ethoxy)-phenyl]-ethane-1,2-diamine (D27)

The title compound was prepared from D4 and D9 using the method described in Description 24. MH⁺ 438/440/442.

### Description 28

### N-(3,4-Dichloro-phenyl)-N′-[4-methoxy-3-(2-morpholin-4-yl-ethoxy)-phenyl]-ethane-1,2-diamine (D28)

The title compound was prepared from D4 and D11 using the method described in Description 24. MH⁺ 440/442/444.

### DESCRIPTION 29

### N-(3,4-Dichloro-phenyl)-N′-[3-(2-dimethylamino-ethoxy)-4-methoxy-phenyl]-ethane-1,2-diamine (D29)

The title compound was prepared from D4 and D13 using the method described in Description 24. MH⁺ 398/400/402.

### DESCRIPTION 30

### N-(3,4-Dichloro-phenyl)-N′-[6-(2-piperidinyl-1-yl-ethoxy)-pyridin-3-yl]-ethane-1,2-diamine (D30)

The title compound was prepared from D4 and D15 using the method described in Description 24. MH⁺ 409/411/413.

### DESCRIPTION 31

### N-[4-Bromo-3-(2-piperidin-1-yl-ethoxy)-phenyl]-N′-(3,4-dichloro-phenyl)-ethane-1,2-diamine (D31)

The title compound was prepared from D4 and D18 using the method described in Description 24. MH⁺ 485/487/489/491

### DESCRIPTION 32

### N-(2,3-Dichloro-phenyl)-N′-[4-methoxy-3-(2-piperidin-1-yl-ethoxy)-phenyl]-ethane-1,2-diamine (D32)

The title compound was prepared from D5 and D9 using the method described in Description 24. MH⁺ 438/440/442

### DESCRIPTION 33

### 1-(3-Benzyloxy-4-methoxy-phenyl)-3-(3-fluoro-phenyl)-imidazolidin-2-one (D33)

Phosgene solution (˜20% in toluene, 2.1 mL, 4.3 mmol) was added dropwise at room temperature to a solution of D24 (1.48 g, 4.0 mmol) in tetrahydrofuran (55 mL) and triethylamine (1.7 mL 12 mmol). After stirring for 2.5 hours the solvent was removed in vacuo and the residue partitioned between ethyl acetate and 2M sodium hydroxide solution. The organic phase was washed with brine, dried over anhydrous magnesium sulphate and concentrated in vacua. The residue was purified by silica gel chromatography to give the title compound as a white solid (1.16 g, 73%). MH⁺ 414.

### DESCRIPTION 34

### 1-(3-Benzyloxy-4-methoxy-phenyl)-3-(4-methyl-3-trifluoromethyl-phenyl)-imidazolidin-2-one (D34)

The title compound was prepared from D25 using the method described in D33. MH⁺ 457.

### DESCRIPTION 35

### 1-(3-Benzyloxy-4-methoxy-phenyl)-3-(2-chloro-phenyl)-imidazolidin-2-one (D35)

The title compound was prepared from D26 using the method described in D33. MH⁺ 430/432.

### DESCRIPTION 36

### 1-(3-Fluoro-phenyl)-3-(3-hydroxy-4-methoxy-phenyl)-imidazolidin-2-one (D36)

D33 (0.79 g, 2.0 mmol) was dissolved in ethanol (70 mL) and N,N-dimethylformamide (30 mL). 10% palladium on charcoal (0.34 g) was added and the mixture hydrogenated at atmospheric pressure for 20 hours. The mixture was filtered through a plug of celite and the solvent removed in vacuo to give the title compound as a grey solid (0.58 g, 96%). MH⁺ 303.

### DESCRIPTION 37

### 1-(4-Methyl-3-trifluoromethyl-phenyl)-3-(3-hydroxy-4-methoxy-phenyl)-imidazolidin-2-one (1)₃₇)

D34 (0.56 g, 1.2 mmol) was partially dissolved in methanol (70 mL) and N,N-dimethylformamide (4 mL). Ammonium formate (0.41 g, 6.5 mmol) and 10% palladium on charcoal (0.1 g) were added and the mixture heated at reflux for 4 hours and 10 minutes. The mixture was allowed to cool to room temperature, filtered through a plug of celite and concentrated in vacuo. The residue was partitioned between dichloromethane (75 mL) and water (75 mL) The organic phase was washed with further water (×2) and brine, dried over anhydrous magnesium sulphate and concentrated in vacuo to give the title compound as a pink solid (0.41 g, 92%). MH⁺ 367.

### Descriptions 38 and 39

### 1-(2-Chloro-phenyl)-3-(3-hydroxy-4-methoxy-phenyl)-imidazolidin-2-one (D38)

### 1-(3-Hydroxy-4-methoxy-phenyl)-3-phenyl-imidazolidin-2-one (D39)

A mixture of D35 (0.350 g, 0.86 mmol), ammonium formate (0.25 g, 4 mmol) and 10% palladium on charcoal (0.035 g) in methanol (20 mL) and N,N-dimethylformamide (1 mL) was heated at reflux for one hour. The mixture was allowed to cool to room temperature, filtered through a plug of celite and concentrated in vacuo. The residue was partitioned between dichloromethane and water. The organic phase was dried over anhydrous magnesium sulphate and concentrated in vacuo. The crude mixture was purified by silica gel chromatography to give the two title compounds D38 and D39. D38 was obtained as a white foam (0.044 g, 16%) MH⁺ 319/321 and D39 was obtained as a cream solid (0.092 g, 38%) MH⁺ 285.

### DESCRIPTION 40

### 1-[3-(2-Benzyloxy-ethoxy)-4-methoxy-phenyl]-3-(3-fluoro-phenyl)-imidazolidin-2-one (D40)

Benzyl 2-bromoethyl ether (0.15 mL, 0.95 mmol) was added to a mixture of D36 (0.26 g, 0.87 mmol), potassium carbonate (0.15 g, 1.1 mmol) and sodium iodide 0.017 g, 0.1 mmol) in N,N-dimethylformamide (10 mL) and heated at 60° C. for 16 hours. The solvent was removed in vacuo and the residue partitioned between ethyl acetate (60 mL) and saturated sodium bicarbonate solution (50 mL). The organic phase was washed with brine (25 mL), dried over anhydrous magnesium sulphate and concentrated in vacuo. The brown oil was triturated with hexane (×3) to give the title compound as a light brown solid (0.34 g, 91%). MH⁺ 437.

### DESCRIPTION 41

### 1-(3-Fluoro-phenyl)-3-[3-(2-hydroxy-ethoxy)-4-methoxy-phenyl]-imidazolidin-2-one (D41)

D40 (0.34 g, 0.79 mmol) was dissolved in ethanol (30 mL) and N,N-dimethylformamide (4 mL). 10% Palladium on charcoal (0.05 g) was added and the mixture hydrogenated at atmospheric pressure for 16 hours. The mixture was filtered through a plug of celite and the solvent removed in vacuo. This gave the title compound as a white solid (0.18 g, 66%). MH⁺ 347.

### DESCRIPTION 42

### Methanesulfonic acid 2-{5-[3-(3-fluoro-phenyl)-2-oxo-imidazolidin-1-yl]-2-methoxy-phenoxy]ethyl ester (D42)

Methanesulfonyl chloride (0.04 mL, 0.53 mmol) was added dropwise at 0° C. to a solution of D41 (0.175 g, 0.5 mmol) in dichloromethane (16 mL) and triethylamine (0.11 mL, 0.76 mmol). The mixture was allowed to warm to room temperature and stirred for 1 hour and 15 minutes. The mixture was washed with saturated sodium bicarbonate solution (40 mL) and brine (40 mL). The organic phase was dried over anhydrous magnesium sulphate and concentrated in vacuo to give the title compound as a white solid (0.208 g, 97%). MH⁺ 425.

### DESCRIPTION 43

### 3-(3-Fluoro-phenyl)-1-(3-hydroxy-4-methoxy-phenyl)-imidazolidine-2,4-dione (D43)

The title compound was prepared from D23 using the method described in Description 36. MH⁺ 317.

### DESCRIPTION 44

### 5-Nitro-3H-benzoxazol-2-one (D44)

2-Amino-4-nitrophenol (26.1 g, 169 mmol) was dissolved in N,N-dimethylformamide (220 mL) and 1,1′-carbonyldiimidazole (30.2 g, 186 mmol) added. The reaction mixture was heated at 80° C. for three hours. The mixture was allowed to cool to room temperature and poured into water (1 Litre) and stirred. The solid was filtered off, washed with water and dried in vacuo to give the title compound as a pale yellow solid. MH⁻ 179.

### DESCRIPTION 45

### 5-Nitro-3-(2-piperidin-1-yl-ethyl)-3H-benzoxazol-2-one (D45)

D44 (0.36 g, 2 mmol) was dissolved in N,N-dimethylformamide (10 mL) and sodium hydride (60% dispersion in oil, 0.20 g, 8.3 mmol) added portionwise. The reaction mixture was stirred at room temperature for one hour and a solution of 1-(2-chloroethyl)piperidine monohydrochloride (0.37 g, 2 mmol) in N,N-dimethylformamide (2 mL) added dropwise. The reaction mixture was stirred at room temperature for sixteen hours. The solvent was removed in vacuo and the residue partitioned between dichloromethane and saturated sodium bicarbonate solution. The organic phase was dried over anhydrous magnesium sulphate and concentrated in vacuo. The residue was purified by silica gel chromatography to give the title compound (0.35 g, 60%). MH⁺ 292.

### DESCRIPTION 46

### 5-Amino-3-(2-piperidin-1-yl-ethyl)-3H-benzoxazol-2-one (D46)

D45 was dissolved in ethanol and 10% palladium on charcoal added. The reaction mixture was hydrogenated at atmospheric pressure for six hours. The mixture was filtered through a plug of celite and the solvent removed in vacuo to give the title compound as an oil (0.29 g, 95%). MH⁺ 262.

### DESCRIPTION 47

5-[2-(3,4-Dichloro-phenylamino)-ethylamino]-3-(2-piperidin-1-yl-ethyl)-3H-benzoxazol-2-one) (D47)

The title compound was prepared from D4 and D46 using the method described in Description 24. MH⁺ 449/451/453.

### EXAMPLE 1

### 1-(3-Fluoro-phenyl)-3-[4-methoxy-3-(2-piperidin-1-yl-ethoxy)-phenyl]-imidazolidin-2-one (E1)

A mixture of D36 (0.1 g, 0.33 mmol) and 1-(2-chloroethyl)piperidine monohydrochloride (0.67 g, 0.36 mmol) in ethylene glycol dimethyl ether (7 mL), water (3 mL) and saturated potassium carbonate solution (2 mL) was heated at reflux for 5 hours. The mixture was diluted with saturated sodium bicarbonate solution (20 mL) and extracted with ethyl acetate (40 mL). The organic phase was washed with brine (20 mL), dried over anhydrous magnesium sulphate and concentrated in vacuo. The residue was purified by silica gel chromatography to give the title compound as a cream coloured solid (0.072 g, 53%).

¹H NMR (CDCl₃) δ: 7.50-7.45 (2H, m), 7.31-7.28 (2H, m), 6.85-6.76 (3H, m), 4.19 (2H, t, 6.3 Hz), 3.94 (4H, s), 3.85 (3H, s), 2.84 (2H, t, 6.3 Hz), 2.54 (4H, bs), 1.62 (4H, m), 1.26 (2H, m). MH⁺ 414.

### EXAMPLE 2

### 1-(3-Fluoro-phenyl)-3-[4-methoxy-3-(2-morpholin-4-yl-ethoxy)-phenyl]-imidazolidin-2-one (E2)

The title compound was prepared from D36 and 4-(2-chloroethyl)morpholine hydrochloride heating for 10 hours using the method described in Example 1.

¹H NMR (CDCl₃) δ: 7.57 (1H, d, 2.5 Hz), 7.49-7.46 (2H, m), 7.32-7.27 (2H, m), 6.86 (1H, d, 8.7 Hz), 6.80-6.76 (2H, m), 4.19 (2H, t, 6.0 Hz), 3.94 (4H, s), 3.85 (3H, s), 3.73 (4H, t, 4.6 Hz), 2.85 (2H, t, 6.0 Hz), 2.60 (4H, t, 4.6 Hz). MH⁺ 416.

### EXAMPLE 3

### 1-(4-Methyl-3-trifluoromethyl-phenyl)-3-[4-methoxy-3-(2-piperidin-1-yl-ethoxy)-phenyl]-imidazolidin-2-one (E3)

The title compound was prepared from D37 and 1-(2-chloroethyl)piperidine monohydrochloride heating for 10 hours using the method described in Example 1.

¹H NMR (CDCl₃) δ: 7.77 (1H, d, 2.0 Hz), 7.70 (1H, dd, 8.4 Hz, 2.1 Hz), 7.26 (1H, m), 6.85 (2H, s), 4.20 (2H, t, 6.3 Hz), 3.93 (4H, s), 3.84 (3H, s), 2.85 (2H, t, 6.3 Hz), 2.55 (4H, bs), 2.44 (3H, d, 1.1 Hz), 1.62 (4H, m), 1.45 (2H, m). MH⁺ 478.

### EXAMPLE 4

### 1-(4-Methyl-3-trifluoromethyl-phenyl)-3-[4-methoxy-3-(2-morpholin-4-yl-ethoxy)-phenyl]-imidazolidin-2-one (E4)

The title compound was prepared from D37 and 4-(2-chloroethyl)morpholine hydrochloride heating for 12 hours using the method described in Example 1.

¹H NMR (CDCl₃) δ: 7.76 (1H, d, 2.0 Hz), 7.71 (1H, dd, 2.0 Hz, 8.4 Hz), 7.56 (1H, d, 2.4 Hz), 7.26 (1H, m), 6.86 (1H, d, 8.8 Hz), 6.79 (1H, dd, 8.8 Hz, 2.4 Hz), 4.19 (2H, t, 6.0 Hz), 3.95 (4H, s), 3.84 (3H, s), 3.73 (4H, t, 4.4 Hz), 2.85 (2H, t, 6.0 Hz), 2.60 (4H, t, 4.4 Hz), 2.44 (3H, d, 0.8 Hz). MH⁺ 480.

### EXAMPLE 5

### 1-(2-Chloro-phenyl)-3-[4-methoxy-3-(2-piperidin-1-yl-ethoxy)-phenyl]-imidazolidin-2-one (E5)

The title compound was prepared from D38 and 1-(2-chloroethyl)piperidine hydrochloride heating for 3 hours and then stirring at room temperature for 16 hours using the method described in Example 1.

¹H NMR (CDCl₃) δ: 7.63 (1H, d, 2.0 Hz), 7.48-7.43 (2H, m), 7.33 (1H, m), 7.26 (2H, m), 6.84-6.82 (2H, m), 4.23 (2H, t, 6.0 Hz), 4.02-3.91 (4H, m), 3.84 (3H, s), 2.91 (2H, broad s), 2.62 (4H, bs), 1.66 (4H, bs), 1.46 (2H, bs). MH⁺ 430/432

### EXAMPLE 6

### 1-[4-Methoxy-3-(2-piperidin-1-yl-ethoxy)-phenyl]-3-phenyl-imidazolidin-2-one (E6)

The title compound was prepared from D39 and 1-(2-chloroethyl)piperidine hydrochloride heating for 3 hours and then stirring at room temperature for 16 hours using the method described in Example 1.

¹H NMR (CDCl₃) δ: 7.64-7.56 (2H, m), 7.37, (2H, m), 7.09 (1H, t, 7.4 Hz), 6.89-6.84 (2H, m), 4.25 (2H, t, 6.0 Hz), 3.99-3.90 (4H, m), 3.85 (3H, s), 2.93 (2H, t, 6.0 Hz), 2.65 (4H, bs), 1.68 (4H, bm), 1.48 (2H, bm). MH⁺ 396

### EXAMPLE 7

### 1-[3-(2-Dimethylamino-ethoxy)₄-methoxy-phenyl]-3-(3-fluorophenyl)-imidazolidin-2-one (E7)

Dimethylamine solution (2M) in tetrahydrofuran (0.5 mL, 1 mmol) and D42 (0.1 g, 0.24 mmol) in N,N-dimethylformamide (7 mL) with potassium carbonate (0.065 g, 0.47 mmol) and sodium iodide (0.035 g, 0.23 mmol) were heated in a bomb at 80° C. for 16 hours. The solvent was removed in vacuo and the residue partitioned between ethyl acetate (50 mL) and saturated sodium bicarbonate solution (20 mL). The organic phase was washed with water (10 mL) and brine (30 mL), dried over anhydrous magnesium sulphate and concentrated in vacuo. The residue was purified by silica gel chromatography to give the title compound as a pale yellow foam (0.074 g, 84%).

¹H NMR (CDCl₃) δ: 7.52-7.46 (2H, m), 7.31-7.29 (2H, m), 6.87-6.77 (3H, m), 4.15 (2H, t, 6.0 Hz), 3.93 (4H, s), 3.85 (3H, s), 2.79 (2H, t, 6.0 Hz), 2.35 (6H, s). MH⁺ 374.

### EXAMPLE 8

### 1-(3-Fluoro-phenyl)-3-{4-methoxy-3[(1S,4S)-2-(2-oxa-5-aza-bicyclo[2.2.1]hept-5-yl)-ethoxy]-phenyl}-imidazolidin-2-one (E8)

(1S,4S)-(+)-2-Aza-5-oxabicyclo[2.2.1]heptane hydrochloride (0.064 g, 0.47 mmol) and D17 (0.1 g, 0.24 mmol) in N,N-dimethylformamide (10 mL) with potassium carbonate (0.081 g, 0.59 mmol) and sodium iodide (0.035 g, 0.23 mmol) were heated at 80° C. for 16 hours. The solvent was removed in vacuo and the residue partitioned between ethyl acetate (60 mL) and saturated sodium bicarbonate solution (15 mL). The organic phase was dried over anhydrous magnesium sulphate and concentrated in vacuo. The residue was purified by silica gel chromatography to give the title compound as an orange oil (0.051 g, 50%).

¹H NMR (CDCl₃) δ: 7.54 (1H, d, 2.4 Hz), 7.49-7.46 (1H, m), 7.32-7.29 (2H, m), 6.86 (1H, d, 8.8 Hz), 6.82-6.76 (2H, m), 4.40 (1H, s), 4.15 (2H, t, 6.4 Hz), 4.08 (1H, d, 8.0 Hz), 3.94 (4H, s), 3.85 (3H, s), 3.66-3.62 (2H, m), 3.10-3.02 (3H, m), 2.64 (1H, d, 10 Hz), 1.88 (1H, d, 10 Hz), 1.74 (1H, d, 10 Hz). MH⁺ 428.

### EXAMPLE 9

### 1-(3,4-Dichloro-phenyl)-3-[4-methoxy-3-(2-piperidin-1-yl-ethoxy)-phenyl]-imidazolidin-2-one (E9)

Phosgene solution (˜20% in toluene, 0.34 mL, 0.7 mmol) was added dropwise at room temperature to a solution of D27 (0.30 g, 0.7 mmol) in tetrahydrofuran (15 mL) and triethylamine (0.34 mL, 2.4 mmol) and stirred at room temperature for 16 hours. Ethyl acetate (120 mL) was added and the reaction mixture washed with 2M sodium hydroxide solution (×3) and brine (60 mL). The organic phase was dried over anhydrous magnesium sulphate and concentrated in vacuo. The crude compound was purified by silica gel chromatography to give the title compound as a beige solid (0.219 g, 69%).

¹H NMR (CDCl₃) δ: 7.72 (1H, d, 2.5 Hz), 7.50 (1H, dd, 8.8 Hz, 2.5 Hz), 7.47 (1H, d, 2.0 Hz), 7.40 (1H, d, 9.1 Hz), 6.87-6.84 (2H, m), 4.18 (2H, t, 6.3 Hz), 3.97-3.87 (4H, m), 3.85 (3H, s), 2.83 (2H, t, 6.3 Hz), 2.52 (4H, m), 1.61 (4H, m), 1.45 (2H, m). MH⁺ 464/466/468.

### EXAMPLE 10

### 1-(3,4-Dichloro-phenyl)-3-[4-methoxy-3-(2-morpholin-4-yl-ethoxy)-phenyl]-imidazolidin-2-one (E10)

The title compound was prepared from D28 using the method described in Example 9.

¹H NMR (CDCl₃) δ: 7.72 (1H, d, 2.4 Hz), 7.54-7.48 (2H, m), 7.40 (1H, d, 8.8 Hz), 6.86 (1H, d, 8.8 Hz), 6.80 (1H, dd, 2.4 Hz), 4.19 (2H, t, 6.0 Hz), 3.97-3.90 (4H, m), 3.85 (3H, s), 3.73 (4H, t, 4.8 Hz), 2.85 (2H, t, 6.0 Hz), 2.60 (4H, t, 4.8 Hz). MH⁺ 466/468/470.

### EXAMPLE 11

### 1-(3,4-Dichloro-phenyl)-3-[3-(2-dimethylamino-ethoxy)-4-methoxy-phenyl]-imidazolidin-2-one (E11)

The title compound was prepared from D29 using the method described in Example 9.

¹H NMR (CDCl₃) δ: 7.72 (1H, d, 2.4 Hz), 7.50-7.47 (2H, m), 7.38 (1H, d, 8.8 Hz), 6.84 (2H, m), 4.15 (2H, t, 6.0 Hz), 3.95-3.88 (4H, m), 3.84 (3H, s), 2.79 (2H, t, 6.0 Hz), 2.35 (6H, s). MH⁺ 424/426/428.

### EXAMPLE 12

### 1-(3,4-Dichloro-phenyl)-3-[6-(2-piperidin-1-yl-ethoxy)-pyridin-3-yl]-imidazolidin-2-one (E12)

The title compound was prepared from D30 using the method described in Example 9.

¹H NMR (CDCl₃) δ: 8.10-8.06 (2H, m), 7.73 (1H, d, 2.8 Hz), 7.50 (1H, dd, 8.8 Hz, 2.8 Hz), 7.40 (1H, d, 8.8 Hz), 6.81-6.78 (1H, m), 4.43 (2H, t, 6.0 Hz), 3.96 (4H, m), 2.76 (2H, t, 6.0 Hz), 2.51 (4H, m), 1.60 (4H, m), 1.44 (2H, m). MH⁺ 435/437/439.

### EXAMPLE 13

### 1-[4-Bromo-3-(2-piperidin-1-yl-ethoxy)-phenyl]-3-(3,4-dichloro-phenyl)-imidazolidin-2-one (E13)

The title compound was prepared from D31 using the method described in Example 9.

¹H NMR (CDCl₃) δ: 7.72 (1H, d, 2.5 Hz), 7.59 (1H, d, 2.5 Hz), 7.51-7.39 (3H, m), 6.77 (1H, dd, 8.7 Hz, 2.5 Hz), 4.17 (2H m), 3.90 (4H, m), 2.86 (2H, m), 2.58 (4H, m), 1.62 (4H, m), 1.44 (2H, m). MH⁺ 512/514/516/518.

### EXAMPLE 14

### 1-(2,3-Dichloro-phenyl)-3-[4-methoxy-3-(2-piperidin-1-yl-ethoxy)-phenyl]-imidazolidin-2-one (E14)

The title compound was prepared from D32 using the method described in Example 9.

¹H NMR (CDCl₃) δ: 7.61 (1H, d, 2.4 Hz), 7.44 (1H, dd, 8.0 Hz, 1.6 Hz), 7.36 (1H, dd, 1.6 Hz, 8.0 Hz), 7.27 (1H, m), 6.85 (1H, 8.8 Hz), 6.80 (1H, m), 4.16 (2H, t, 6.4 Hz), 4.00 (2H, m), 3.90 (2H, m), 3.85 (3H, s), 2.82 (2H, 6.4 Hz), 2.50 (4H, m), 1.58 (4H, m), 1.43 (2H, m). MH⁺ 464/466/468.

### EXAMPLE 15

### 1-(3-Fluoro-phenyl)-3-[4-methoxy-3-(2-piperidin-1-yl-ethoxy)-phenyl]-1,3-dihydro-imidazol-2-one (E15)

D20 (0.110 g, 0.23 mmol) in toluene (12 mL)was treated with concentrated hydrochloric acid (one drop) and the mixture heated at reflux for three hours. The mixture was diluted with ethyl acetate and washed with saturated sodium bicabonate solution (25 mL) and brine (40 mL). The organic phase was dried over anhydrous magnesium sulphate and concentrated in vacuo. The crude compound was purified by silica gel chromatography to give the title compound (0.049 g, 52%).

¹H NMR (CDCl₃) δ: 7.52-7.49 (1H, m), 7.42-7.39 (2H, m), 7.30 (1H, d, 2.4 Hz), 7.05 (1H, dd, 2.4 Hz, 8.4 Hz), 6.96 (1H, m), 6.91 (1H, d, 8.4 Hz), 6.71 (2H, dd, 12.8 Hz, 3.2 Hz), 4.19 (2H, t, 6.4 Hz), 3.88 (3H, s), 2.84 (2H, t, 6.4 Hz), 2.52 (4H, m), 1.60 (4H, m), 1.45 (2H, m). MH⁺ 412.

### EXAMPLE 16

### 1-(3,5-Difluoro-phenyl)-3-[4-methoxy-3-(2-piperidin-1-yl-ethoxy)-phenyl]-1,3-dihydro-imidazol-2-one (E16)

The title compound was prepared from D21 using the method described in Example 15.

¹H NMR (CDCl₃) δ: 7.34-7.30 (2H, m), 7.25 (1H, m), 7.03 (1H, dd, 8.6 Hz, 2.5 Hz), 6.92 (1H, d, 8.6 Hz), 6.73-6.68 (3H, m), 4.18 (2H, t, 6.4 Hz), 3.88 (3H, s), 2.83 (2H, t, 6.4 Hz), 2.52 (4H, m), 1.59 (4H, m), 1.44 (2H, m). MH⁺ 430.

### EXAMPLE 17

### 1-(3,5-Difluoro-phenyl)-3-[4-methoxy-3-(2-piperidin-1-yl-ethoxy)-phenyl]-imidazol-2-one (E17)

E16 (0.064 g, 0.15 mmol) was dissolved in ethanol (15 mL) and 10% palladium on charcoal added (0.02 g). The reaction mixture was hydrogenated at atmospheric pressure for 48 hours. The mixture was filtered through a small plug of celite and the solvent removed in vacuo. The residue was triturated with petroleum ether (40-60° C.) (×3) to give the title compound (0.035 g, 55%).

¹H NMR (CDCl₃) δ: 7.45 (1H, d, 1.7 Hz), 7.22-7.17 (2H, m), 6.88-6.85 (2H, m), 6.52 (1H, m), 4.18 (2H, t, 6.4 Hz), 3.97-3.88 (4H, m), 3.85 (3H, s), 2.83 (2H, t, 6.4 Hz), 2.51 (4H, m), 1.62 (4H, m), 1.46 (2H, m). MH⁺ 432.

### EXAMPLE 18

### 3-(3-Fluoro-phenyl)-1-[4-methoxy-3-(2-piperidin-1-yl-ethoxy)-phenyl]-imidazol-2,4 dione (E18)

Diethyl azodicarboxylate (0.055 g, 0.32 mmol) in tetrahydrofuran (1 mL) was added dropwise to a stirred solution of D43 (0.1 g, 0.32 mmol), triphenylphosphine (0.083 g, 0.32 mmol) and N-(2-hydroxyethyl)piperidine (0.041 g, 0.32 mmol) in tetrahydrofuran (4 mL) at room temperature. After stirring for 24 hours the reaction mixture was passed through an ion exchange SCX cartridge eluting with methanol and 0.5 M ammonia/methanol. The ammonia fractions were combined and concentrated in vacuo. The residue was purified by silica gel chromatography followed by trituration with petroleum ether (40-60° C.) (×2) to give the title compound as a white solid. (0.062 g, 54%).

¹H NMR (CDCl₃) δ: 7.50-7.45 (2H, m), 7.32-7.24 (2H, m), 7.15-7.02 (1H, m), 6.87 (2H, m), 4.45 (2H, s), 4.17 (2H, t, 6.4 Hz), 3.87 (3H, s), 2.83 (2H, t, 6.4 Hz), 2.51 (4H, m), 1.60 (4H, m), 1.45 (2H, m). MH⁺ 428

### EXAMPLE 19

### 5-[3-(3,4-Dichloro-phenyl)-2-oxo-imidazolidin-1-yl]-3-(2-piperidin-1-yl-ethyl)-3H-benzooxazol-2-one (E19)

The title compound was prepared from D47 using the method described in Example 9.

¹H NMR (CDCl₃) δ: 7.73 (1H, d, 2.4 Hz), 7.71 (1H, d, 2.4 Hz), 7.50 (1H, m), 7.42 (1H, d, 8.8 Hz), 7.16 (1H, d, 8.8 Hz), 6.92 (1H, dd, 8.8 Hz, 2.4 Hz), 4.04-3.93 (6H, m), 2.67 (2H, t, 6.8 Hz), 2.47 (4H, bm), 1.54 (4H, bm), 1.41 (2H, bm). MH⁺ 475/477/479.

1. A compound of formula (I): 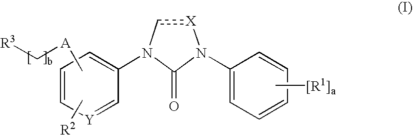
wherein:
a is 0, 1, 2,3,4 or 5;
b is 1, 2 or 3;
Y is nitrogen or carbon;
A is oxygen, nitrogen, —CONH—, —NHCO— or together with R² form a benzoxazolone group;
R¹ is halogen, C₁₋₆alkyl, C₁₋₆alkoxy, C₁₋₆alkylthio, hydroxy, amino, mono- or di-C₁₋₆alkylamino, nitro, CN, CF₃, OCF₃, aryl, arylC₁₋₆alkyl, arylC₁₋₆alkyloxy or arylC₁₋₆alkylthio;
R² is hydrogen, halogen, C₁₋₆alkyl, C₁₋₆alkoxy, haloC₁₋₆alkyl or haloC₁₋₆alkoxy;
R³ is:
(i) —NR⁴R⁵ where R⁴ and R⁵ are independently hydrogen, C₁₋₆alkyl or arylC₁₋₆alkyl; or
(ii) an optionally substituted N-linked heterocycle; or
(iii) an optionally substituted C-linked heterocycle; 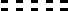
is a single bond or a double bond;
X is CH₂ or C═O (when 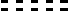
is a single bond) or X is CH (when 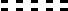
is a double bond).

2. A compound as claimed in claim 1, wherein a is 1 or 2.

3. A compound as claimed in claim 1 or claim 2, wherein R¹ is halogen.

4. A compound as claimed in claim 1, 2 or 3, wherein R¹ is at the 3 position or the 4 osition.

5. A compound as claimed in any of claims 1 to 4, wherein X is CH₂ and 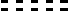
represents a single bond or X is CH and 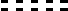
represents a double bond.

6. A compound as claimed in any of claims 1 to 6, wherein Y is carbon.

7. A compound as claimed in any of claims 1 to 6, wherein R² is at the following position:

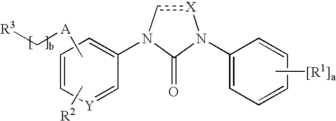

8. A compound as claimed in any of claims 1 to 7, wherein R² is C₁₋₆alkoxy.

9. A compound as claimed in any of claims 1 to 8, wherein A is at the following position:

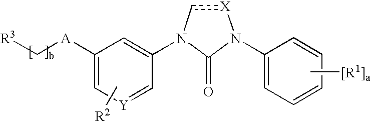

10. A compound as claimed in any of claims 1 to 9, wherein A is oxygen.

11. A compound as claimed in any of claims 1 to 10, wherein b is 2.

12. A compound as claimed in any of claims 1 to 11, wherein R³ is N-linked heterocycle or diC₁₋₆alkylamine.

13. A compound as claimed in claim 1, which is:
1-(3-Fluoro-phenyl)-3-[4-methoxy-3-(2-piperidin-1-yl-ethoxy)-phenyl]-imidazolidin-2-one;
1-(3-Fluoro-phenyl)-3-[4-methoxy-3-(2-morpholin-4-yl-ethoxy)-phenyl]-imidazolidin-2-one;
1-(4-Methyl-3-trifluoromethyl-phenyl)-3-[4-methoxy-3-(2-piperidin-1-yl-ethoxy)-phenyl]-imidazolidin-2-one;
1-(4-Methyl-3-trifluoromethyl-phenyl)-3-[4-methoxy-3-(2-morpholin-4-yl-ethoxy)-phenyl]-imidazolidin-2-one;
1-(2-Chloro-phenyl)-3-[4-methoxy-3-(2-piperidin-1-yl-ethoxy)-phenyl]-imidazolidin-2-one
1-[4-Methoxy-3-(2-piperidin-1-yl-ethoxy)-phenyl]-3-phenyl-imidazolidin-2-one;
1-[3-(2-Dimethylamino-ethoxy)-4-methoxy-phenyl]-3-(3-fluorophenyl)-imidazolidin-2-one;
1-(3-Fluoro-phenyl)-3-{4-methoxy-3 [(1S,4S)-2-(2-oxa-5-aza-bicyclo[2.2.1]hept-5-yl)-ethoxy]-phenyl}-imidazolidin-2-one;
1-(3,4-Dichloro-phenyl)-3-[4-methoxy-3-(2-piperidin-1-yl-ethoxy)-phenyl]-imidazolidin-2-one;
1-(3,4-Dichloro-phenyl)-3-[4-methoxy-3-(2-morpholin-4-yl-ethoxy)-phenyl]-imidazolidin-2-one;
1-(3,4-Dichloro-phenyl)-3-[3-(2-dimethylamino-ethoxy)₄-methoxy-phenyl]-imidazolidin-2-one;
1-(3,4-Dichloro-phenyl)-3-[6-(2-piperidin-1-yl-ethoxy)-pyridin-3-yl]-imidazolidin-2-one;
1-[4-Bromo-3-(2-piperidin-1-yl-ethoxy)-phenyl]-3-(3,4-dichloro-phenyl)-imidazolidin-2-one;
1-(2,3-Dichloro-phenyl)-3-[4-methoxy-3-(2-piperidin-1-yl-ethoxy)-phenyl]-imidazolidin-2-one;
1-(3-Fluoro-phenyl)-3-[4-methoxy-3-(2-piperidin-1-yl-ethoxy)-phenyl]-1,3-dihydro-imidazol-2-one;
1-(3-Fluoro-phenyl)-3-[4-methoxy-3-(2-piperidin-1-yl-ethoxy)-phenyl]-1,3-dihydro-imidazol-2-one;
1-(3-Fluoro-phenyl)-3-[4-methoxy-3-(2-piperidin-1-yl-ethoxy)-phenyl]-imidazol-2-one;
3-(3-Fluoro-phenyl)-1-[4-methoxy-3-(2-piperidin-1-yl-ethoxy)-phenyl]-imidazol-2,4-dione;
5-[3-(3,4-Dichloro-phenyl)-2-oxo-imidazolidin-1-yl]-3-(2-piperidin-1-yl-ethyl)-3H-benzoxazol-2-one,
or a pharmaceutically acceptable salt thereof.

14. A process for the preparation of a compound of formula (I) or a pharmaceutically acceptable salt thereof, which process comprises:
a) the cyclisation of a compound of formula (II) 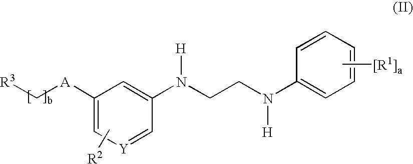
in which R¹, R², R³, A, Y, a and b are defined in formula (I); or
b) the coupling of a compound of formula (III): 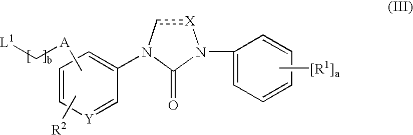
in which R¹, R², A, X, Y, a and b are defined in formula (I) and L¹ is a leaving group, with a compound of formula (IV):
H—NR⁴R⁵ (IV)
in which R⁴ and R⁵ are defined in formula (I); or
c) the coupling of a compound of formula (V) 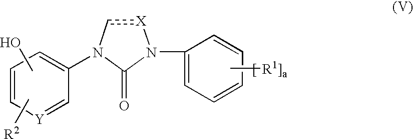
in which R¹, R², X, Y and a are defined in formula (I), with a compound of formula (VI): 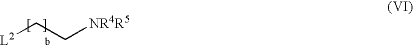
in which R⁴, R⁵ and b are defined in formula (I) and L² is a leaving group or hydroxy; or
d) the cyclisation of a compound of formula (VII): 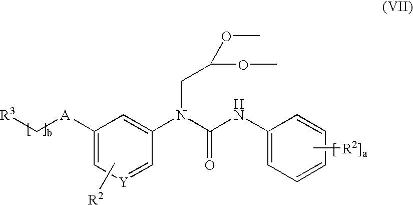
in which R¹, R², R³, Y, A, a and b are defined in formula (I);
optionally followed by:
removing any protecting groups; and/or
converting a compound of formula (I) so formed into another compound of formula (I);
and/or
forming a pharmaceutically acceptable salt.

15. A pharmaceutical composition comprising a compound as defined in any of claims 1-13 and a pharmaceutically acceptable carrier or excipient.

16. A process for preparing a pharmaceutical composition as defined in claim 15, the process comprising mixing a compound as defined in any of claims 1-13 and a pharmaceutically acceptable carrier or excipient.

17. A compound as claimed in any of claims 1-13 or a composition as claimed in claim 15 for use as a therapeutic substance.

18. A compound or a composition as defined in claim 17 for use in the treatment of a CNS disorder.

19. A compound or a composition as defined in claim 17 wherein the CNS disorder is depression and/or anxiety.

20. A method of treatment of a CNS disorder in mammals, comprising administering to the sufferer a therapeutically safe and effective amount of a compound as defined in any of claims 1-13 or a composition as defined in claim 15.

21. A method as claimed in claim 20, wherien the CNS disorder is depression and/or anxiety.

22. Use of a compound as defined in any of claims 1-13 or a composition as defined in claim 15 in the manufacture of a medicament for use in the treatment of a CNS disorder.

23. Use as claimed in claim 22, wherein the CNS disorder is depression and/or anxiety.
